(19) United States
(12) Patent Application Publication
(10) Pub. No.: US 2002/0010947 A1
(43) Pub. Date: Jan. 24, 2002

(54) Transgenic mouse model of human neurodegenerative disease

(76) Inventors: Mark E. Gurney, Reykjavik (IS); Irene Abraham, Seattle, WA (US)

Correspondence Address: WOODCOCK WASHBURN KURTZ MACKIEWICZ & NORRIS LLP, ATTENTION: SUZANNE E. MILLER ESQ., ONE LIBERTY PLACE, 46TH FLOOR, PHILADELPHIA, PA, 19103, US

(21) Appl. No.: 09/767,088

(22) Filed: Jan. 22, 2001

Related U.S. Application Data
(60) Provisional application No. 60/177,319, filed on Jan. 21, 2000.

(51) Int. Cl. A01K 67/027

(52) U.S. Cl. 800/12; 800/18

(57) ABSTRACT

This invention provides transgenic mice expressing wild-type or mutant isoforms of human tau protein. The transgenic mice of the invention have many uses, including screening candidate drug compounds effective against neurodegenerative diseases and other diseases involving taupathies. The transgenic mice of the invention can also be used in genetic crosses with other transgenic mice strains to model the progression of neurodegenerative disease.

### CROSS-REFERENCE TO RELATED APPLICATIONS

This application claims priority under 35 U.S.C. §112(e) to U.S. Provisional Patent Application, Ser. No. 60/177,319 filed Jan. 21, 2000, which is hereby incorporated by reference.

### FIELD OF THE INVENTION

This invention is related to the fields of transgenic animals, neurodegenerative disease, and of identifying compounds to treat such diseases.

### BACKGROUND OF THE INVENTION

Many neurological diseases such as Alzheimer's disease, frontotemporal dementia and Parkinsonism linked to chromosome 17 (FTDP-17), progressive supranuclear palsy, corticobasal degeneration, and other undefined dementias, have the common pathological feature of neurofibrillary tangles (NFTs). NFTs are structures made up of paired helical filaments (PHFs), composed primarily of tau protein in a hyperphosphorylated state. Tau belongs to a family of microtubule associated proteins (MAPs) that are thought to play critical roles in stabilizing and remodeling the neuronal cytoskeleton. Under normal physiological conditions tau is found primarily in the brain, associated with the cellular cytoskeleton.

The adult human brain expresses six different isoforms of tau protein, ranging in size from 352 to 441 amino acid residues. The isoforms differ by having either three or four microtubule binding domains, and by the presence or absence of amino-terminal inserts. Tau is phosphorylated in the brain by a variety of kinases, although the relationship between hyperphosphorylation and tangle formation is unclear. It is also unclear how plaque and tangle formation leads to cognitive dysfunction and neuronal loss in patients with Alzheimer's disease.

Insight into the relationship of tau pathology and dementia has come through the study of FTDP-17, which is caused by mutations in the tau gene (see generally Goedert et al., 1998, Neuron 21:955-958). The signature neuropathy associated with FTDP-17 is insoluble filamentous aggregates of hyperphosphorylated tau protein occurring in the absence of amyloid plaque and Lewy bodies, hallmarks of Alzheimer's disease and Pick's disease, respectively. Tau deposits are present not only in neurons, but also in large numbers of glial cells including predominantly oligodendrocytes, and also astrocytes (Spillantini et al., 1997, Proc. Natl. Acad. Sci. USA 94:4113-4118). Many of the tau protein deposits also react with antibodies directed against ubiquitin. Immuno-electron microscopy demonstrates that tau is present as cytoplasmic filaments reminiscent of the paired helical filaments found in Alzheimer's disease, but these helical filaments have a different diameter and periodicity (Spillantini et al., 1998, Am. J. Pathol. 153:1359-1363). Pathology varies between patients and may correlate with the particular tau mutation (Rizzu et al., 1999, Am. J. Hum. Genet. 64:414-421).

Human tau protein has been expressed in transgenic mice in a number of studies. Gotz et al. (1995, EMBO J., 14:1304-1313) used the human Thy-1 promoter to express, in transgenic mice, the longest isoform of wild-type human tau, which is 441 amino acids in length, containing the 58 amino acid amino-terminal insert and four microtubule binding domains. Brion et al. (1999, Am. J. Pathol. 154:255-270) used the murine 3-hydroxy-methyl-glutaryl CoA reductase (HMG-CR) promoter to express the shortest human tau isoform, which is 352 amino acids in length, contains no amino-terminal inserts, and has three microtubule binding domains. An increase in tau phosphorylation at sites associated with Alzheimer's disease pathogenesis is seen in both models, as shown by reactivity with the PHF1 and AT8 monoclonal antibodies that recognize phosphoepitopes on the tau protein. In the HMG-CR/tau mice, this increase in tau phosphorylation is accompanied by the formation of a conformation-dependent epitope associated with early Alzheimer's disease pathology (Brion et al., supra). This epitope is detected with an antibody designated Alz50, raised against paired helical filaments purified from Alzheimer's disease brain. Neurofibrillary tangles were not present in either transgenic model.

Zahr et al. (1999, Soc. Neurosci. 25:(A)447.1) have recently reported transgenic expression in mice of mutant tau, containing the V337M mutation (i.e., the valine at position 337 of the wild-type is replaced with methionine) in the context of a three microtubule binding domain isoform, under the regulatory control of the mouse prion promotor.

Additional transgenic models in which an increase in tau phosphorylation is seen include mice expressing mutant human amyloid precursor protein (APP) (Sturchler-Pierrat et al., 1997, Proc. Natl. Acad. Sci. USA 94:13287-13292), glycogen synthase kinase-3beta (Brownlees et al., 1997, Neuroreport 8:3251-3255), and the serine/threonine kinase Mos (James et al., 1996, Neurobiol. Aging 17:235-241). Filamentous tau pathology is also absent from these models.

Considerable effort has been put into making transgenic models of human neurodegenerative disease based on expression in transgenic mice of pathogenic human mutations. Choice of mutation, choice of transgene vector, expression in appropriate types of neurons, and transgenic expression of the mutant human protein at sufficient levels are all critical to success. Strategies for creation of transgenic models have therefore varied widely. For example, the transgenic models for familial amyotrophic lateral sclerosis used the 12 Kb SOD1 gene for expression of human Cu,Zn superoxide dismutase containing the mutations G93A (Gurney et al., 1994, Science 264:1772-1775) or G37R (Wong et al., 1995, Neuron 14:1105-1116).

Transgenic models for amyloid β-peptide deposition in Alzheimer's disease have used either the platelet-derived growth factor promoter (Games et al., 1995, Nature 373:523-527), insertion into the human prion gene (Hsiao, 1997, J. Neural. Transm. Suppl. 49:135-144), or the human Thy-1 promoter (Sturchler-Pierrat et al., supra) to elicit transgenic expression of the amyloid precursor protein, containing either the “London” or “Swedish” mutations associated with familial, early-onset Alzheimer's disease. These types of transgenic mice are becoming extraordinarily valuable to the pharmaceutical industry for their use as “gatekeeper” models for preclinical evaluation of promising drug candidates. An example of such a use was the testing of riluzole in the transgenic model of amyotrophic lateral sclerosis, where it proved to have a beneficial effect (Gurney et al., 1996, Ann. Neurol. 39:147-157). The drug later became the first to be approved by the Food and Drug Administration for the treatment of the human disease. Similar use is being made of transgenic mice that model various aspects of Alzheimer's disease pathology.

The current transgenic models of Alzheimer's disease, which are based on expression of mutant human amyloid precursor protein, are incomplete models of the disease. Although they show amyloid β-peptide deposition in plaque, no neurofibrillary tangles are noted, nor is there extensive neuronal cell loss or consequent brain atrophy. Thus, the art is in need of new and/or better models of Alzheimer's disease as well as other neurodegenerative diseases. The present invention is directed to addressing these, and other needs.

### SUMMARY OF THE INVENTION

This invention provides transgenic mice expressing wild-type or mutant isoforms of human tau protein. The transgenic mice of the invention can be used for screening candidate drug compounds, which may be effective against neurodegenerative diseases and other diseases involving taupathologies. The transgenic mice of the invention can also be used in genetic crosses with other transgenic mice strains to model the progression of neurodegenerative disease.

In one aspect, the present invention is directed to a transgenic mouse expressing a human tau protein under the regulatory control of the mouse prion gene promoter. The human tau protein that is expressed in the transgenic mouse may be either wild-type or may contain a mutation. The mutation may be one previously identified in humans, or may be a novel mutation. The human tau protein expressed may be any of the six different isoforms.

The invention is also directed to the use of the transgenic mice of the invention as models of neurodegenerative disease.

In another aspect of the invention, the transgenic mice are used to discover drugs that modulate tau protein expression and/or activity.

These and other aspects of the invention are more fully described below.

### BRIEF DESCRIPTION OF THE DRAWINGS

FIG. 1 shows a schematic of a tau transgene construct used to prepare a transgenic mouse of the present invention. See also Table 3, infra.

FIG. 2 shows immunoblot analyses of human tau protein expression in the brains of wild-type and two kinds of mutant tau transgenic mice. Mouse brain samples are identified by mouse transponder ID number (5 digit number) or by ear tag number (337 and 341) (see Table 4, infra). Samples 21080, 21089, 21096, 22410, 22418, and 22435 serve as controls without human tau protein expression, and are from non-transgenic litter mates of the transgenic mice. Mice with transponder numbers are the progeny of the original founder mice mated with (C57B1/6×SJL)F1 partners; mice with three digit numbers are the founder mice.

### DETAILED DESCRIPTION OF THE INVENTION

The invention provides transgenic mice, containing transgenes of human tau, and expressing human tau protein.

Transgenic animals contain within their genetic material some genetic information from another organism. For example, a transgenic mouse might have a human gene inserted into its genetic material. Such foreign genetic material is referred to as a transgene.

Transgenic mice can be generated in a number of manners known to those of skill in the art. Typically, a transgenic animal is generated according to the following basic steps: 1) engineer a nucleic acid construct with a promoter (regulatory sequences) and cDNA or gene sequences of interest; 2) linearize and inject the transgene construct into the pronuclei of embryos; 3) implant the embryos into the uterus of a pseudo-pregnant female; 4) screen the offspring that are born (the live pups in the case of mice) for the presence of the transgene; and 5) breed the transgene-positive pups (founders) to get transgenic “lines.” Examples of these and related techniques are found in, e.g., Hogan, B., Costantini, F. & Lacy, E., eds., Manipulating the Mouse Embryo: A Laboratory Manual, Cold Spring Harbor Laboratory Press, Cold Spring Harbor, N.Y. (1986), which is incorporated herein by reference in its entirety.

Tau is predominantly expressed in neurons; the protein is principally localized in the axons. Some expression has also been noted in oligodendrocytes and astrocytes. Six different isoforms of tau protein are expressed in the adult human brain. Each isoform is generated by differential splicing of an RNA transcript from the single tau gene, located on chromosome 17. The six isoforms range in size from 352 to 441 amino acid residues. The isoforms differ by having either three or four microtubule binding domains (imperfect repeats of 31 or 32 amino acid residues) in the carboxy-terminus, and by the presence or absence of 29- or 58-amino acid inserts, of unknown function, in the amino-terminus.

Tau protein microtubule binding domains, which contain the core microtubule binding domain motif proline-glycine-glycine-glycine (PGGG), are designated R1, R2, R3, and R4, and are encoded by exons 9, 10, 11, and 12, respectively. Exon 10, which encodes amino acid residues 275 through 305, is alternatively utilized (present in three of the six isoforms), such that the R2 microtubule binding domain is present only in tau isoforms containing four repeats. Thus, the isoforms range in size from 352 amino acid residues (with no amino-terminal inserts and three microtubule binding domains) to 441 amino acid residues (with a 58 amino acid amino-terminal insert and four microtubule binding domains). Table 1 summarizes the features of the six human tau isoforms, which are designated “A” through “F.” The isoforms may also be respectively referred to as numbers “1” through “6,” or by their total length in amino acid residues.

TABLE 1
The Six Isoforms of Human Tau Protein:

| | N-Terminal | Microtubule | Length |
|---|---|---|---|
| | Insert | Binding Domains | (amino acids) |
| A | none | 3 | 352 |
| B | 29 amino acids | 3 | 381 |
| C | 58 amino acids | 3 | 410 |
| D | none | 4 | 383 |
| E | 29 amino acids | 4 | 412 |
| F | 58 amino acids | 4 | 441 |

In fetal brain, only the smallest isoform of tau (“A” in Table 1) is expressed. The amino acid residues in tau protein, regardless of isoform, are typically referred to by the position number they would have in the largest isoform (“F”), which has 441 residues. See Spillantini & Goedert, 1998, Trends Neurosci. 21:428-433.

The DFTP-17 associated mutations in tau provide an important entry point in the study of neurofibrillary tangle formation as it relates to neuronal dysfunction and death. Tau mutations linked to FTDP-17 are either missense mutations (see Table 2), leading to substitution of one amino acid for another, or mutations in the 5′ splice site of exon 10.

Most of the missense mutations associated with FTDP-17 occur in or near three of the four homologous microtubule binding domains within tau. Many of the missense mutations reduce the ability of tau to bind microtubules and to promote microtubule assembly in biochemical assays (Hong et al., 1998, Science 282:1914-1917; Hasegawa et al., 1998, FEBS Lett. 437:207-210). The arginine to tryptophan mutation at amino acid 406 (R406W), although linearly distant from a microtubule binding domain (residue 406 is encoded in exon 13, which does not contain a repeat), is believed to interfere with microtubule binding through a conformational change in the protein (Hong et al., supra). The asparagine to lysine at 279 (N279K), proline to leucine at 301 (P301L), and serine to asparagine at 305 (S305N) mutations are seen only in four repeat-containing tau isoform proteins, as these residues are encoded by alternatively spliced exon 10. The valine to methionine at residue 337 (V337M) mutation is of particular interest, because it causes a tau pathology in the brain indistinguishable from that seen in Alzheimer's disease (Spillantini et al., 1996, Acta Neuropathol. 92:42-48; Poorkaj et al., 1998 Ann. Neurol. 43:815-825).

TABLE 2
Tau Missense Mutations Associated with FTDP-17

| | | Location Within | |
|---|---|---|---|
| Codon* | Amino Acid Substitution | Tau Protein | Reference |
| 272 | Glycine → Valine | R1 Microtubule Binding | 1 |
| | (G272V) | Domain | |
| 279 | Asparagine → Lysine | R1 Microtubule Binding | 2, 3, 7 |
| | (N279K) | Domain | |
| 301 | Proline → Leucine | R2 Microtubule Binding | 1, 2, 4, 7 |
| | (P301L) | Domain | |
| 305 | Serine → Asparagine | R2 Microtubule Binding | 3, 8 |
| | (S305N) | Domain | |
| 337 | Valine → Methionine | R3 Microtubule Binding | 5, 6, 7 |
| | (V337M) | Domain | |
| 406 | Arginine → Tryptophan | Carboxy-Terminal to R4 | 1, 7 |
| | (N406W) | Microtubule Binding | |
| | | Domain | |

*Numbering is based on the longest tau isoform, containing 441 amino acid residues.

1. Hutton et al., 1998, Nature 393 :702-705

2. Clark et al., 1998, Proc. Natl. Acad. Sci. USA 95:13103-13107

3. Hasegawa et al., 1999, FEBS Lett. 443:93-96

4. Dumanchin et al., 1998, Hum. Mol. Genet. 7:1825-1829

5. Poorkaj et al., supra (refers to codon based on numbering of a shorter tau isoform)

6. Spillantini et al., 1998, Brain Pathol. 8:387-402

7. Hong et al., supra

8. lijima et al., 1999, Neuroreport 10:497-501

Two studies have examined the effect of several tau mutants on microtubule structure after transfection of the tau genes into cells in culture (Dayanandan et al., 1999, FEBS Lett. 446: 228-232; Arawaka et al., 1999, Neuroreport 10:993-997). These studies show variable effects of the mutants on microtubule structure. Arawaka et al. (supra) showed that the presence of the V337M mutation results in fewer cell processes and reduced microtubule networks. Dayanandan et al. (supra) reported no differences in morphology with V337M, P301L or R406W, but did see differences in cell processes after treatment with cytochalasin B. In addition, Nacharaju et al. (1999, FEBS Lett. 447:195-199) have shown that mutants P301L, V337M and R406W cause an accelerated aggregation of tau into filaments in vitro.

A number of splice mutations have been identified in association with FTDP-17. These mutations are located in the 5′ region of the intron immediately following exon 10 of the tau gene. The mutations are: G to A at nucleotide position +3 of the intron (Spillantini et al., 1998, Proc. Natl. Acad. Sci. USA 95:7737-7741); A to G at +13 (Hutton et al., 1998, Nature 393:702-705); C to T at +14 (Hutton et al., supra, Clark et al., supra, Hong et al., supra); and C to T at +16 (Hong et al., supra). These splice mutations all destabilize a potential stem-loop structure that may be involved in regulation of the alternative splicing of exon 10. This causes more frequent usage of the 5′ splice site and an increased proportion of tau transcripts that include exon 10 (Hutton et al., supra). The increase in transcripts containing exon 10 presumably is mirrored by an increase in the proportion of tau protein containing four repeats of the microtubule binding domain. An increase in splicing of exon 10 is also seen with two of the tau missense mutations, asparagine to lysine at residue 279 (N279K) and serine to asparagine at residue 305 (S305N), neither of which alters microtubule binding or assembly (Hasegawa et al., 1999,supra).

Based on what is understood about the mutations associated with FTDP-17, two types of mutations are sufficient to cause tau neuropathology: (1) mutations that alter the ability of tau to bind to microtubules and promote microtubule assembly; and (2) mutations that increase the proportion of tau protein containing four microtubule binding domains.

The present invention provides transgenic mice expressing human tau protein in either wild-type or mutant forms. In one embodiment of the invention, human tau protein is expressed in transgenic mice under the control of regulatory sequences from the mouse prion (PrP) gene promoter region. Preferably such expression is directed to the brain. More preferably, the regulatory sequences include the 5′ flanking sequence of the mouse prion gene promoter, the first exon, the first intron, and the initial noncoding portion of the second exon of the mouse prion gene.

In a preferred embodiment, the human tau protein that is expressed in the transgenic mice is the wild-type 383 amino acid isoform of the protein (isoform D). This isoform contains four microtubule binding domains (i.e., it utilizes exon 10), but has no amino-terminal insert; it is the shortest of the four repeat-containing isoforms.

In another embodiment of the invention, mutant human tau protein is expressed in the transgenic mice of the invention. Preferably, the mutation is the V337M mutation in the 383 amino acid isoform of the human tau protein. Those of skill in the art will recognize that other mutations can be used in the context of any of the six different isoforms of tau.

The tau protein expressed in the transgenic mice of the invention may contain a mutation known to be associated with FTDP-17, including, but not limited to, the G272V, N279K, P301L, S305N, V337M, and R406W missense mutations.

The invention also provides transgenic mice expressing mutant forms of human tau protein, containing mutations other than those known to be associated with FTDP-17. Those of skill in the art will recognize that such mutations can be selected based on the locations of microtubule binding domains and other important features of the tau protein. Mutations to be incorporated into tau transgenes can be chosen based on predicted interference with tau function or analogy with disease-associated mutations. Such mutations can be tested for their effect on tau function by transfection studies on tissue culture cells. Those of skill in the art will recognize that any mutations in tau which modulate tau activity can be used in the transgenic mice of the invention.

Examples of amino acid residues in tau that can be mutated include residue 332, which is a proline in wild-type tau. Residue 332 is located in the core PGGG motif of the R3 microtubule binding domain. Mutation of residue 332 from proline to leucine (P332L), mimics the FTDP-17—associated alteration at residue 301 (P301L) in the core PGGG motif of the R2 microtubule binding domain. In a preferred embodiment of the invention, the mutant human tau expressed in the transgenic mice contains the P332L mutation in the 383 amino acid isoform of the protein.

A further embodiment of the invention includes transgenic mice expressing human tau protein containing a missense mutation at amino acid residue 364, where proline has been changed to leucine, resulting in alteration of the core PGGG motif of the R4 microtubule binding domain, also mimicking the FTDP-17-associated mutation, P301L.

In a preferred embodiment of the invention, the nucleotide sequence encoding the human tau protein is provided by a nucleic acid containing no intronic sequences. Other embodiments may include intronic sequences in the transgene to study the effect of mutations that occur in such intronic regions, for example, the mutation of G to A at position +3 in the intron immediately following exon 10. Thus, the invention also provides transgenic mice containing transgenes of human tau with intronic mutations.

In another embodiment, the transgenic mice of the invention are used as models of the progression of neurodegenerative disease or other diseases whose hallmark is neuronal cell death. Preferably, the transgenic mice of the invention will develop, with age, tau pathologies including tau hyperphosphorylation and filamentous aggregates of tau. Tau hyperphosphorylation may be revealed by immunostaining for tau phosphoepitopes such as those recognized by PHF1 or AT8 monoclonal antibodies. Filamentous tau neuropathology may be revealed by staining for ubiquitin immunoreactivity. More preferably, the tau transgenic mice will display neuronal loss and cognitive dysfunction.

In another embodiment of the invention, transgenic mice expressing tau protein are used to screen for compounds that can be used to treat neurodegenerative disease.

It will be appreciated by those of skill in the art that the transgenic mice of the present invention are useful for analysis of neurodegenerative diseases involving tau pathologies. Such diseases involving tau pathology, collectively referred to as taupathies, include, but are not limited to, Alzheimer's disease, Pick's disease, FTDP-17, progressive supranuclear palsy (PSP), and corticobasal degeneration (CBD), Down's syndrome, Argyrophilic grain disease, amyotrophic lateral sclerosis/parkinsonism dementia complex of Guam, Non-Guamanian motor neurone disease with NFT, Niemann-Pick disease type C, subacute sclerosing panencephalitis, postencephalitic parkinsonism, dementia pugilistica, myotonic dystrophy, Gerstmann-Straeussler-Scheinker disease with tangles, prion protein amyloid antipathy, presenile dementia with tangles and calcifications, and Hallervorden-Spatz disease (Tolnay & Probst, 1999, Neuropathol. Appl. Neurobiol. 3:171-187).

Thus, in another embodiment, the invention is a model of neurodegenerative disease comprising any of the above described transgenic mice. In another embodiment, the transgenic mice of the invention are used in the screening of drug compounds to identify those capable of treating neurodegenerative disease.

In another embodiment, the transgenic mice of the invention are used in the screening of drug compounds to identify those capable of modulating the expression and/or activity of tau protein.

In another embodiment, the transgenic mice of the invention are used to screen for compounds that can be used to treat diseases that involve neuronal cell death.

Other embodiments of the invention will be readily understood by those of skill in the art.

The invention is further illustrated by way of the following examples, which are intended to elaborate several embodiments of the invention. These examples are not intended, nor are they to be construed, as limiting the scope of the invention. It will be clear that the invention may be practiced otherwise than as particularly described herein. Numerous modifications and variations of the present invention are possible in view of the teachings herein and, therefore, are within the scope of the invention.

### EXAMPLES

### Example 1

### Preparation of Transgenic Vectors

Plasmid phgPrP, containing most of the mouse PrP gene 5′ end and its 3′ flanking sequences, was obtained from Marek Fischer, Department of Medicine, Division of Infectious Diseases and Hospital Epidemiology, University Hospital Zurich, Ramistrasse 100, CH-809 Zurich, Switzerland. Plasmid T43, containing the cDNA encoding the wild-type 383 amino acid isoform of human tau [SEQ ID NO:1] in the vector pSG5 (Stratagene, La Jolla Calif.; GenBank Accession AF013258), was obtained from Virginia Lee, Department of Pathology and Laboratory Medicine, University of Pennsylvania School of Medicine, 34th & Spruce Street, Philadelphia, Pa. 19104.

The sequence of the phgPrP insert was confirmed by the DNA Sequencing Core (Pharmacia & Upjohn). The 14,214 base pair (bp) phgPrP insert sequence was verified by comparison with the 38,418 bp GenBank sequence deposit for the Mus musculus short incubation prion protein Prnpa gene (Accession Number U29186). The phgPrP insert sequence extends the GenBank deposit by 5,047 bp of 5′ flanking sequence [SEQ ID NO:2]. The entire phgPrP insert was cloned into pBluescript KS+ (Stratagene, La Jolla, Calif.). The resulting construct was opened with KpnI to remove the PrP coding sequence and 3′ flanking region of the mouse PrP gene. Amplification by polymerase chain reaction (PCR) was used to resynthesize the 3′ mouse PrP flanking sequence such that it contained 5′ KpnI and XhoI sites for cloning. This created the vector 97-1, used for subsequent steps in assembly of the PrP/tau transgene.

To assemble the PrP/tau transgene, the 97-1 vector was opened with XhoI and SalI. This retained the mouse PrP promoter and sequences through the initial, noncoding portion of PrP exon 2. SailI was used to excise the human tau-383 insert fused to the polyadenylation site from the pSG5 vector. After dephosphorylation of the 97-1 vector, the two DNAs were ligated and transformed into E. coli strain DH5α (GIBCO/BRL, Gaithersberg, Md.). Bacterial colonies were screened for directional ligation of the tau insert in the sense orientation by PCR using the primers AGTAATTGAAAGAGCTCAGACGATG [SEQ ID NO:3] and TGTCACCCTCTTGGTCTTGGTGC [SEQ ID NO:4].

The V337M mutation was introduced into the tau-383 coding sequence by mutagenesis of a 302 bp PstI-HindIII DNA fragment using PCR. The PCR reaction was carried out using: a primer positioned 5′ of the PstI site having the sequence GTACTCCACCCAAGTCGCCGTC [SEQ ID NO:5], a short 3′ reverse primer containing the HindIII site having the sequence GCAGCAGCATCGAAGCTTCTCAG [SEQ ID NO:6], and a longer reverse “patch” primer containing the V337M mutation having the sequence GCAGCAGCATCGAAGCTTCTCAGATTTTACTTCCATCTGGCCACCTCC [SEQ ID NO:7].

The P332L mutation was also introduced into the tau-383 coding sequence by mutagenesis using PCR. The PCR reaction was carried out using a forward primer having the sequence CCGCCAAGAGCCGCCTGCAG [SEQ ID NO:8], the SEQ ID NO:6 reverse primer, and a longer reverse “patch” primer containing the P332L mutation, having the sequence GCAGCAGCATCGAAGCTTCTCAGATTTTACTTCCACCTGGCCACCTCCTAGTTTATGA TGG [SEQ ID NO:9].

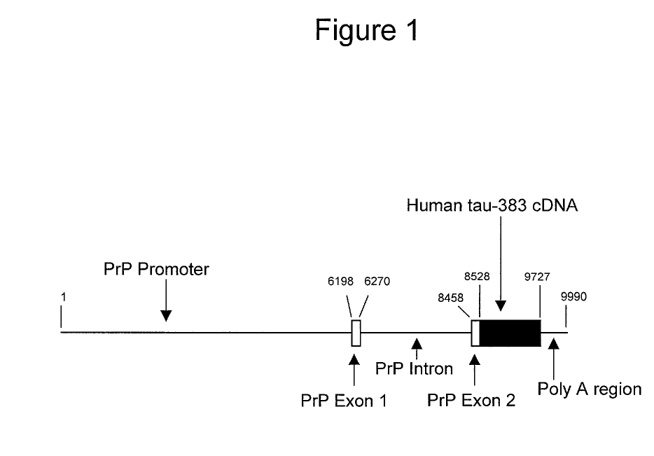

The PCR reaction was performed with an Advantage-HF™ high-fidelity PCR kit (Clontech, Palo Alto, Calif.), using the buffer supplied with the kit. The “patch” PCR primer was used at 1/100th the concentration of the short, flanking primers. Five cycles of (95° C. for 45 seconds, 60° C. for 2 minutes, 72° C. for 3 minutes) followed by 20 cycles of (95° C. for 45 seconds, 68° C. for 3 minutes) were used for synthesis of the mutant fragment. To construct a mutant tau-383 insert, the PstI-HindIII fragment from wild-type tau-383 was exchanged for a mutant fragment. Proper introduction of mutations was confirmed by sequencing in the Pharmacia & Upjohn DNA sequencing core. The cDNA sequence for the V337M mutant of the 383 amino acid isoform is provided in SEQ ID NO:10. The cDNA sequence for the P332L mutant of the 383 amino acid isoform is provided in SEQ ID NO:11. The same procedure as described above for wild-type tau was used to assemble mutant inserts, tau-V337M and tau-P332L, into the PrP transgene vector. Table 3 presents the general features in the transgene constructs used to generate tau transgenic mice. The features correspond to those depicted in FIG. 1. The sequence for the entire 9990 nucleotide sequence of the PrP/tau transgene vector comprising wild-type 383 tau is provided in SEQ ID NO:15.

TABLE 3
General Features in the PrP/tau Transgene Vectors
Used for Microinjection

| Feature | Nucleotide Location |
|---|---|
| PrP promoter | 1-6197 |
| | (Nucleotides 1-5047 are designated |
| | SEQ ID NO:2) |
| PrP exon 1 | 6198-6270 |
| PrP intron 1 | 6271-8457 |
| PrP exon 2 | 8458-8527 |
| tau-383 cDNA | 8528-9727 |
| poly(A) region | 9728-9990 |

### Example 2

### Generation and Screening of Transgenic Mice

The 10 Kb plasmid inserts were prepared for microinjection by the University of Michigan Transgenic Facility following standard protocols. They were injected into F2 fertilized mouse eggs from matings of F1 hybrid C57B1/6×SJL mice. Progeny were genotyped by Taqman™ PCR using mouse genomic DNA prepared from tail biopsies using the Qiagen tissue kit according to manufacturer's instructions. Amplification was performed with the Taqman™ PCR Core Reagents Kit (PE Biosystems) using 50 mM KCl, 10 mM Tris-HCl, pH 8.3, 10 mM EDTA, 60 nM Passive Reference Dye 1, 5 mM MgCl₂, 200 μM each of dATP, dGTP and dCTP, 400 μM dUTP, 200 nM each primer, 100 nM Taqman probe, 0.5 units uracil-N-glycosylase, and 1.25 units AmpliTaq Gold. Specific amplification of human tau DNA was performed using the primers G29-Fp GCATTGGAGACACCCCCAG [SEQ ID NO:12], G29-Rp GCTTTTACTGACCATGCGAGC [SEQ ID NO:13], and G29 Taqman™ probe with 5′ and 3′ calorimetric tags FAM-CTGGAAGACGAAGCTGCTGGTCACG-TAMRA [the nucleotide portion of which is designated SEQ ID NO: 14] with the thermal cycling parameters, 50° C.-2 minutes, 95° C.-10 minutes, 40 cycles of [95° C.-15 seconds, 60° C.-1 minute], and 25° C. hold. The starting amount of mouse genomic DNA template was 10 ng. A standard curve was generated ranging 5 orders of magnitude from 102 to 106 copies of linearized PrP/tau transgene plasmid DNA, which was diluted into mouse genomic DNA to give a final concentration of 10 ng/μl. All reactions were performed in duplicate.

Multiple mice were generated from fertilized mouse eggs injected with wild-type or mutant constructs. For each construct, several founder mice, carrying the tau transgene, were identified. Table 4 shows that the transgenic mice contained varying numbers of copies of the PrP/tau transgene, from which varying levels of transcript were expressed.

TABLE 4
Summary of Analysis of Transgenic Tau Mice

| | | | htau RT-PCR | htau RT-PCR |
|---|---|---|---|---|
| | | | levels copies/ | levels copies/ |
| Transgene | | | 67 ng total | 67 ng total |
| and founder | Mouse | Gene | RNA positive | RNA negative |
| mouse number | Id | Copy # | RT sample | RT sample |
| TgN(PrP/tau) 355 | 21088 | 4.6 | 16,599 | 81 |
| TgN(PrP/tau) 327 | 21097 | 2.6 | 5,402 | 13 |
| TgN(PrP/tau) 326 | 21083 | 13.7 | 595 | 161 |
| | 21214 | 10.6 | 754 | 26 |
| TgN(PrP/tau- | 22419 | 2.4 | 33,671 | 6 |
| V337M) 249 | 22420 | 2.6 | 26,912 | 20 |
| TgN(PrP/tau- | 22405 | 0.3 | 19,138 | 0 |
| V337M) 250 | 22408 | 0.2 | 21,814 | 0 |
| TgN(PrP/tau- | 22436 | 0.9 | 10,755 | 2 |
| V337M) 257 | 22437 | 0.2 | 8,842 | 1 |
| TgN(PrP/tau- | 337 | 16.8 | ND¹ | ND |
| P332L) 337 | | | | |
| TgN(PrP/tau- | 341 | 1.4 | ND | ND |
| P332L) 341 | | | | |

¹Not determined.

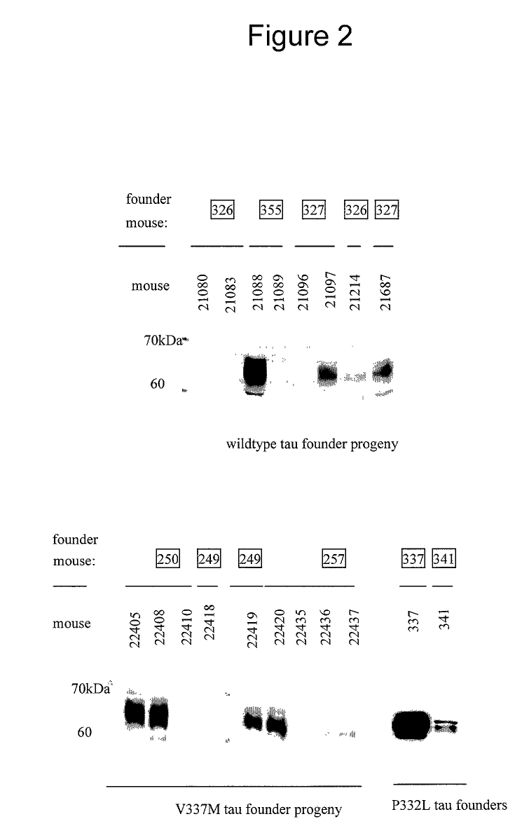

Table 4 summarizes the analysis of the transgenic mice. The first column lists the transgene designation (TgN) and founder mouse number. The founder mice are indicated by the numbers 355, 327, 326, 249, 250, 257, 337 or 341, after the transgene name TgN(PrP/tau), TgN(PrP/tau-V337M) or TgN(PrP/tau-P332L). The second column lists the mouse identification (Id) number (either a 5-digit transponder number or a 3-digit founder number, as used in FIG. 2). Mice with a 5-digit number are the progeny of the founder mouse indicated in the first column by the three digit number after the transgene name. The third column lists the haploid copy number of the human tau (htau) transgene, based on DNA levels derived after PCR analysis. This number was measured either in the founder or founder progeny, as indicated in the second column. The fourth column lists the human tau (htau) transgene mRNA expression, as determined by reverse transcriptase-PCR (RT-PCR). The fifth column lists the background amplification of the RNA samples, without conversion to cDNA by RT.

### Example 3

### Human Tau Expression

Mouse brain samples were harvested after intracardiac perfusion with cold phosphate-buffered saline, for analysis of human tau mRNA and tau protein expression. For cardiac perfusion, mice were deeply anesthetized, then perfused transcardially until the effluent was cleared of blood. Tissue samples were frozen immediately on dry ice, then stored at −70° C. until analysis.

Total RNA was prepared from homogenized cortical brain tissue using the RNeasy mini kit (Qiagen). The RNeasy protocol was modified by the addition of the RNase-free DNase set protocol (Qiagen) to treat the RNA, while bound onto the column, with RNase-free DNase I at room temperature for 15 minutes to 1 hour, to remove any residual genomic DNA contamination. Two micrograms of total RNA was converted to cDNA in a 30 μl reaction containing, 5 mM Tris-HCl, pH 8.3, 75 mM KCl, 3 mM MgCl₂, 500 μM each dNTP, 0.5 μg random hexamers, 30 units RNase inhibitor, and 400 units Superscript II reverse transcriptase (RT), which was incubated at 42° C. for 1 hour. The Superscript II RT enzyme was then heat inactivated by incubation at 95° C. for 5 minutes. To assess the extent of genomic DNA contamination in the total RNA samples, control reactions, without RT, were also performed for each sample.

For determination of human tau mRNA expression, PCR amplification on the ABI PRISM 7700 instrument was performed using 2X Taqman™ universal PCR master mix (PE Biosystems) containing 200 nM G29Fp and 200 nM G29Rp primers and 100 nM G29 Taqman probe. The final PCR reaction volume was 25 μl, and contained 5 μl of a 1:5 dilution of the cDNA reaction described above. The thermal cycling parameters were as follows, 50° C.-2 minutes, 95° C.-10 minutes, 40 cycles of [95° C.-15 seconds, 60° C.-1 minute], and 25° C. hold. A relative standard curve was generated ranging 5 orders of magnitude from 102 to 106 copies of linearized PrP/tau transgene plasmid DNA, which was diluted into mouse genomic DNA to a final concentration of 10 ng/μl. All samples were run in either duplicate or triplicate.

For determination of human tau protein expression by immunoblot analysis, brain tissue was minced in 600 μl lysate buffer (0.1M MES, pH 6.8, 0.75M NaCl, 0.5 mM EGTA, 2 mM DTT, 2 mM PMSF, 10 μg/ml aprotinin, 10 μg/ml leupeptin, 10 μ/ml soy bean trypsin inhibitor, 5 μg/ml pepstatin) on ice. Brain fragments were ground with 10 strokes of a pestle in a pre-chilled TenBroeck tissue homogenizer. Lysate was centrifuged at 40,000 g for 15 minutes at 4° C. Protein concentration of supernatants was determined by Bio Rad protein assay, and lysates were stored at −80° C. until used.

50 μg samples of the brain lysates were diluted into 4X gel loading buffer (250 mM Tris-HCl, pH 6.8, 8% SDS, 20 mM EDTA, 40% glycerol, 0.02% bromophenol blue, 20% β-mercaptoethanol), heated at 100° C. for 5 minutes, and electrophoretically separated on a 7.5% polyacrylamide gel in running buffer (25 mM Tris base, 192 mM glycine, 0.01% SDS). The proteins were transferred to Nitrobind nitrocellulose membrane (Micron Separations Inc, Westborough, Mass.) in transfer buffer (25 mM Tris base, 192 mM glycine, 20% methanol). The membranes were blocked for 1 hour in PBS containing 5% nonfat dry milk. Blots were probed overnight at 4° C. with either the T14 or HT7 anti-human tau antibody (Zymed Laboratories, Inc., So. San Francisco, Calif.), diluted to 2 μg/ml in a solution of 10 mM Tris-HCl, pH 7.5, 500 mM NaCl, and 0.1% Tween 20 (TNTween), plus 1% BSA and 3% dry milk. After washing in TNTween, the blots were incubated for 1 hour at room temperature with goat anti-mouse, horseradish peroxidase-conjugated secondary antibody (Kirkegaard and Perry Laboratories, Inc., Gaithersburg, Md.) at 1:5000 dilution. The immunoblots were washed again in TNTween, treated with ECL reagents for 1 minute, and exposed to X-ray film.

Progeny of the transgenic founders (mated to F1 hybrid (C57B1/6×sSJL) partners) or the founders were analyzed for PrP/tau transgene expression in the brain. Both human tau mRNA (Table 4) and protein (FIG. 2) were detected. Efficient, relatively high levels of human tau protein expression were obtained in transgenic mice using a mouse PrP gene vector coupled to a heterologous human tau-383 cDNA. Roughly equivalent levels of protein expression were obtained from wild-type and mutant PrP/tau transgenes, suggesting that expression of mutant tau protein is relatively well tolerated in the mouse. The levels of expression achieved appear to be higher than those reported by Gotz et al., supra or Brion et al., supra, although different antibodies and methods of analysis were used to establish levels of protein expression. Ranking by protein expression roughly correlates with the PRISM measurement of mRNA expression.

### Example 4

### Microtubule Organization in GFP-tau Transfected Cells

To investigate the effect of mutations in the microtubule binding domains on tau protein interaction with microtubules, human embryonic kidney HEK293 cells were transiently transfected with cDNA encoding the 383 human tau isoform fused to green fluorescent protein (GFP). Wild-type and the P332L, V337M, and P301L tau mutants were analyzed. Fluorescence and biochemical analyses showed that GFP-labeled mutant tau proteins P332L, V337M, and P301L have an altered intracellular distribution, as compared to GFP-wild type tau protein.

Studies using an anti-tubulin antibody revealed that GFP-wild-type tau protein co-localizes with microtubules. Some tau protein is also present in the cytoplasm, not associated with microtubules. In cells expressing high levels of wild-type tau, a reorganization of the microtubule cytoskeleton was observed. Bundles of microtubules could be seen, often in the form of a spiral or a ring around the circumference of the cell. This is a phenomenon previously noted in non-neuronal cells expressing tau. Cells expressing low levels of GFP-wild type tau protein, contained a fine network of microtubules, often emerging from microtubule organizing center.

Cells expressing GFP-labeled mutant tau proteins P332L, V337M, and P301L exhibited higher levels of fluorescence dispersed in the cytoplasm than did cells expressing GFP-wild type tau protein. The microtubule bundling and the microtubule network, seen in GFP-wild type tau expressing cells, was also reduced in cells expressing the mutant tau proteins.

Immunoblotting analyses of cytosolic and cytoskeletal cellular fractions confirmed the fluorescence studies, showing that cells expressing the GFP-mutant tau proteins contained more tau protein in the cytosol than did cells expressing GFP-wild type tau. The difference was most dramatic for P332L, but the studies have confirmed that these microtubule binding domain mutations interfere with tau protein interaction with microtubules.

### Example 5

### Cellular Analysis of Tau Transgenic Mice

General pharmacokinetic studies are used to analyze human tau expression in the transgenic mice. Pharmacokinetic studies include determination of the brain distribution of tau protein expression, and kinase activity assays. The state of phosphorylation of the tau protein is detected using monoclonal antibodies to specific phosphoepitopes of tau, including PHF1 and AT8, and various polyclonal antibodies. Filamentous tau neuropathology is revealed by staining for ubiquitin immunoreactivity.

### Example 6

### Analysis of Neurodegenerative Disease in Transgenic Mice

Neurodegenerative disease is studied in tau transgenic mice by use of a variety of behavioral studies, including the Morris water maze test, mobility and gait tests, and behavioral observation (see, e.g., Moechars et al., 1999, Biol. Chem. 274:6483-6492).

### Example 7

### Drug Screening in Transgenic Mice

The transgenic mice are used for the screening of compounds that affect tau hyperphosphorylation, filament formation,or neurodegenerative disease criteria.

Compounds that inhibit kinase activity, protease activity, phosphatase activity, oxidative stress reactions, apoptosis, or protein aggregation are tested for their ability to modulate 1) tau phosphorylation, 2) tau aggregate formation, 3) formation of cellular inclusions of paired helical filaments of tau, and 4) formation of neurofibrillary tangles in the transgenic mice. Those compounds exhibiting positive effects in such tests may be useful in the treatment of neurodegenerative diseases.

All references cited herein are incorporated herein in their entirety by reference.

We claim:

1. A transgenic mouse, comprising a transgene, said transgene comprising a polynucleotide encoding a human tau protein operably linked to at least a portion of a regulatory region of a mouse prion gene, wherein said transgenic mouse expresses human tau protein.

2. The transgenic mouse of claim 1, wherein said human tau protein is selected from the group consisting of isoforms 352, 381, 410, 383, 412, and 441.

3. The transgenic mouse of claim 2, wherein said human tau protein is isoform 383.

4. The transgenic mouse of claim 1, wherein said human tau protein is selected from the group of mutants consisting of G272V, N279K, P301L, S305N, V337M, and R406W.

5. The transgenic mouse of claim 4, wherein said human tau protein is a V337M mutant of isoform 383.

6. The transgenic mouse of claim 2, wherein said human tau protein is further selected from the group of mutants consisting of G272V, V337M, and R406W.

7. The transgenic mouse of claim 3, wherein said human tau protein is further selected from the group of mutants consisting of N279K, P301L, and S305N.

8. The transgenic mouse of claim 2, wherein said human tau protein is mutant P332L.

9. The transgenic mouse of claim 1, wherein said human tau protein is expressed in the brain.

10. The transgenic mouse of claim 1, wherein said regulatory region comprises said prion gene promoter and 5′ flanking sequence, the first PrP exon, the first PrP intron, and the initial, noncoding portion of the second PrP exon.

11. A model of neurodegenerative disease comprising a transgenic mouse as in any of claims 1-10.

12. The model of neurodegenerative disease of claim 11 for use in the screening of drugs to treat said disease.

13. The model of neurodegenerative disease of claim 11 for use in genetic crosses to generate models of Alzheimer's disease.

14. A method of screening for a drug that modulates hyperphosphorylation of tau comprising the steps of
a) administering the drug to a transgenic mouse, the transgenic mouse comprising a transgene, said transgene comprising a polynucleotide encoding a human tau protein operably linked to at least a portion of a regulatory region of a mouse prion gene, wherein the transgenic mouse expresses hyperphosphorylated human tau protein; and
b) comparing the state of phosphorylation of tau in a second transgenic mouse to which the drug was not administered to the state of phosphorylation of tau in the transgenic mouse to which the drug has been administered, wherein a difference in phosphorylation indicates the drug modulates hyperphosphorylation of tau.

15. A method of screening for a drug for treatment of a neurodegenerative disease comprising the step of administering the drug to a transgenic mouse which exhibits neurodegenerative disease characteristics, the transgenic mouse comprising a transgene, said transgene comprising a polynucleotide encoding a human tau protein operably linked to at least a portion of a regulatory region of a mouse prion gene, wherein the transgenic mouse expresses human tau protein, and wherein it is determined whether the drug at least partially abates at least one of the characteristics of the disease.

16. A method of screening for a drug that blocks hyperphosphorylation of tau comprising the step of administering the drug to a transgenic mouse, the transgenic mouse comprising a transgene, said transgene comprising a polynucleotide encoding a human tau protein operably linked to at least a portion of a regulatory region of a mouse prion gene, wherein the transgenic mouse expresses hyperphosphorylated human tau protein, and wherein it is determined whether the drug at least partially blocks hyperphosphorylation of tau in the transgenic mouse.

17. A method of screening for a drug that blocks formation of filamentous aggregates of tau comprising the step of administering the drug to a transgenic mouse, the transgenic mouse comprising a transgene, said transgene comprising a polynucleotide encoding a human tau protein operably linked to at least a portion of a regulatory region of a mouse prion gene, wherein the transgenic mouse expresses human tau protein forming filamentous aggregates, and wherein it is determined whether the drug at least partially blocks formation of filamentous aggregates of tau in the transgenic mouse.


SEQUENCE LISTING (15 sequences)

SEQ ID NO: 1 - DNA, 1152 bp, Homo sapiens
atggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtga

SEQ ID NO: 2 - DNA, 9990 bp, Mus musculus
ggcggccgcgacggatccaaaggcagcaaaaaggcagagagggtgatactgggcctggcttaagcatttgaaacttcaaagctcacccccaattacacacttcttccaacaagtccacacctcctaattagtgccactctctgtgggcctacggagagtattttcattctaactaccacagttgctgaggaatttaattaaaactacaaccttatcccaacctagatctttcagcctttctgtactaccagagaggggtcatacagcattgttgtgactcccattataacttaaagggaagctcacacaaagtccagagccctccataccctgcaaatgaagaagtacgttctcaaatcccttggagcagggccccactttggcggcacaaactttaatttctagacggaacggcatctctacagaaagaaaagccatggtatctgcatgataagtctgaaaaggacctgggcaaatctgcagctgacaattccagccattgctgccactgcgagaaaaccctgctgatggcagcattgtcagcatcatctcctccaggaacaccggccatcgagccacgaggacaattgctgctgctggagtcaattcatctgccagccacatcatactctgggaccgtcactaaccagatccaagcagccttgaggaagcatgtcttctggtggtgactgatcccaagggctgacaacaaggtcctcacagaggcatcttatgtcaacctatctaccatgcacggtataagacacattctcctctgtgctgtgtggacactgccatcacacgcaacagaaaggaaactcactcactgtgtctgatgtggtggtgcttgttaggggagttctgggcatgtatggcaccatcgcccatgaggactcctgtggggtcatgcccactctactcctctagagaccatgaagagatggagagggaagagcaagcacagatgacaggctagaactaaagaggagtgtcaggtgagcggacctgaactcacggctgctcagcctgaagtggtgtggccatctgcatctggtatctggtctgaaggtgcgtggataccctctgtgcccgtccagaagtttcctactgaagacagaaatgcctgtccagtcatggaagaatgattggcagttcccacttctcagaccactgaatgggtcagaacaactactgggtgaccctaaggtattcttcagcagatatgtgtgaaaaatggaaagaagatgggtagaaataaacggttttagaggaaaaaaactctcacaaagatattataaaaagaaaagagctttattattgagcaagcattcaaccagaatgcacaccacaggcagtctgctaagggagtgtgcagacaggaggagtgtcgccctttatgtgagccagtagataaggatgctgtgcgtgtttttagtaactggtcttcagcttgacagcaccatttatcacatggtttaacctaaattcatctggcgaatgaggctgtcacgtacttcctgattagctttatctgaaatgagacaagcttcacatgttcacggcaggaggtaatcctgctgcttagagaacagggtccatccaagccaggctccttctcccaccaacacgggtggttgaagagctatctctccctggtgtgtgtgtttcagagatggctcccaggtttttggtttggtttgaattgggttttggttttcttactctagcccagactagcttggaattctctggaaagctgcaacggggagctcaggttcagtgagagatcctgtctcaaaaagcagggtgagaagtgattgaggaagacaccccagtgttaacctctgacctccatatgtgcatgcatggacacgcatggatacacatacacacacacacacacacacacacacacacacacacacacacacacacacaaaaccagaaagaatgaacgcccccctcccagcttgtttacagtagatacagagcactcgtaaaacatggggtgtaaactgaatgctgagagtaacttagatgagtaattaaggaaggaagaggaaagaaaccaggaaaccgagagcaagtgactggaagatcgttaggcaatctccacaccctgctcgttgaagttggaatgctttcttcttctgcctcttgaagttctttagaagtgctaggatttcacaattagtctgtggtggtttcaatatgcttcacccgtggtaagtggcactattaggaaacgtgtccttgttgaaggaagggtgtcactgcataggcgggctttgaggtgtcttccagtgctcaagctcctcccagtgcaagagaggcagacacctgttgcctgcagaagacagtctcctgctgcctttgaatcaagatgtagaactcaagccccatgtctgcctgaacctctgaaactgtaagccagccccaattaaatgttttctttcacaagagttgccttggtcatggtgtctgttcacagcaataaaaccctaactaagacagtcttaaatcaatgaaaagacctttaattattcattgaacaaacaccattttcttgtatcaagttggcagtgactagtaagcaactatagttctgcaccagggacctttttggagaaatataccgatccaagcatgttggcatctagattccaaagccaagacacctgccacacccttccatgccttgggttcctggcagggcatctggcttcggggatgtgtattccaggcacccactggaatgcatggaaacaattaaaatagcatcatagaagacattgcaatcctagggagaaactataccaaaactcagaactatacctggttaagtgtagaaaagacgaaaggaataaaaccaggaatattttaaaatatttttattgagctcatgtgcatgggtattttgcctgaaagtatgtctgtgtaccacatgcatggctggctcctgcagaggccaaaagagagcatcagatctcctagagctggagtttcagaagtttgtgagctaccacatgggtgctggaaacaaaacccaggacatctggaagagcagccagtgttcttaactactgagccatcactcaggtcccaccatgaatgtttttctttattcttctctatattttctaatgtttttattggaaatatacaacttttgccacacataacaaatgaccaaagaaatgaggtgagaggggcagctgttcaaatgctgcctgggaaggcttggccagccctggcttggctgcccctggctcagctggccctgacttggctgtcccggtgccagctgtcatctactgcttcataataagctgcactttgggctgaaggggtggctcagcctttaaaggctaggctcataaccaaagtaagttgcattttatttgcactaggttgaagggggatctgaaacttgctgtcaatgttataaaacattttatcttcaaatttggtataggggtcatagaccaaaggttctataaaccccagaacagcaccactccctagaaataagcacccatacaagagcctatgggacactttatagccaaacaaaaagctatgtttgaaacttcctttacaagggcctgagtcccattcataagggaaggagccccacttcgtaataacaccccactggtgacatttgaaggggacacattcaaactgtaacaccatcttatatcatttgcacattagggtcaaactgtgccacgttgtcatttctaagaagacagaagttgtcaagcctgtgctttgagccacaagtgtgacaacctactttcaggcaagtcgctacttccctaagactctaccccaataggcctggggtctggaatgtgtttaacacagatgcaggcttctgccttagtgcaggcttgagttctcatgtccctctctctttagctttccgtctcaaggcgcctctccttagcagaaaaaatcagaggcataaagcatacatcagggggaagccagagttttcagagggagttttgtgatggccttttcagagcattcttgtcaagactagtttgcctcgttctctttattaaatgaaagaaaaataatgcagtgttgcaaattagctttggtaatggctccaaccattgtcaggttcacagtctcattccgccattcaaaacaacaaacccaccacactctctatgcagtgccgtaactcagaacagccaccaaacagcagaaagaggctccccgactcctctcagccttgccataaactcgccggccacatgcttattttaaattatttaaattatgtcgtttctcccaacaatgacctcccaagtgcttggttgacaggcttataccattaagccgaggcttgcatagcaacgataaccaggtaggctattattataaccaggtagctgccgagctactggtcggtccccttttgtctctagaaacctctcaacccccacccaaaaaagcttttattgccacttcctagtgggtagagagcagtcagccaatagatatttgattctttgaggaaaaagctgagttttgatgtcttttaatcaagcctttcagagtccctctgtgggggaggccaggtggaagcggggtgggaagctggtcccttacctaagctaatctagacaccctcccactcctcccctgccctcttgacagatgcagtcatcctgatcacaatgagtattctctgaggcaggaaggcaaggctctggaagatggtcaatgccttcattaagaacccagagtaaaggtcaagcagacaccagcaccgctgaaatctaatttcactgtaattgaatcatctcagccaaaggctgtattttccagccctctcgtggcctcttccccaacaactgtcaacaactgtgtgagcctacccatgtatgcgcgctcacacacacacacacacacacacacacacacacacacagggtggggggacacaatgattacacaagagtacttaataaacaactataattctcctggctcggatagttccttaccaccctctcctcctggatccggatcctaatactggatacaaatatttaatccaaacccaatcttgtgtctgttaatgatcttcagtgtctcgccctcagcaagaggacaggatattatgttttccctgtgatttatgacctcttctgtctcagtatcggcagcaatttatttacatggctttggagtgtgttatatgtgtagtatggacatgagggtgcatgtcaacctatgtgtggaggccagaggtcaatgtcatgtcttccccaatcactgcccagtggtccctggattccaaactcaggtcctcacgcttgggaactgagccagtgccccagctcctaaccctcccctgttttaaaaaggtctcattatgttgcccaggtcagccttgaacttgagagtctcctgactgcaggctttcacctgtccaagtcagcaggcatcttgaacaagaacatcatttcctttaagctgtttcaggctgtgtttggtgggagctgttaaatgcagtgcattttttcctttggacacaataaaagaaaaaagtgattaaatgagttgggtgtggtggtgcgagtctacaatcctagaactcaggagattgagggagaagcattgctctgagtttgaggtcagcttaaattacttagtaggaacaccaggccaaattgggctatgggattgtctccaaagataaagaaaaaagggaaggagagaaaagaaaaagaaaggaaagaaggggaaaagaaggaatcagcagagaataaataagtcaacatgcaatggccaatatactttctaggcctctaattcttttatagtttgtgggaaaatgtcgaaaatcttcgttaccaatttcttgttaccaaagttcaacgatggcttcctcgctccgttaggtaacctttcattttctcaactacccattatgtaacgggagcattgggtactggatcagtcttccattaaagatgatttttatagttgctgagcgtcgtcagggagtgctgacactgggggcggtttaaacagatacaagcatttaagccagtccggagcggtgactcatccccccccacccccacccccccgcgagagacgcggcgcggccattggtgagcatcacgccccgcccctcgccccgcctagttcccgcctgccccgcccctttccactcccggctcccccgcgttgtcggatcagcagaccgattctgggcgctgcgtcgcatcggtggcaggtaagcgggctgctgaagccaggccttggcgagcactcagccttccgtcgtcaagctcggctcactgcgcctctcggggccttgaggccacggggactaggactgggactgggactggggctgagtctggctgggaggtgactgtacaccccctgctgcgcgactcctggaggaaccgaatcccagggcagccaggccgggagccagcctttccttcccgagccagattcacagctcagcatcgctggggatgggggtggcatcttttgactgtccttggctgttttcttctctctttgtagtagctacagcgaacataattttacctcgttattccaccacagtcattactcccttgcacagtttcattctcaacgtcgccgtgcgccttcactgccctgtctaggcgttttcatgattgtctattttcttgtactttgaataccgtggtttaatagcagttgcgggtgcgcagaattctccatttccttaagagaaactcctgggagaatgggactaaagacgtgcaaatttaattatatcgcaaacaggaatcaaaattttgcattaaaatgccaaacatcttgaaaaattaactattcaatgaagaaaaggaactactttacctacacacacatccgagagcttcgaggaggcgaaggaaatagaaagctaagggatgatttgggttgtatttgaatctgacacaagctttccatattatttatagcagggactaaacgatgagtcattttctgaataagatgcaaattaaagcaagtttgtttgttgtctttacatctattaaatagacagagacaatggcaacagcaaccctaacctagaggttgcctgaaagtgtcaggtttgggaacaagtggccctgcttaagggctagaaagattgctttacaaccaacaatcatgacttgacattgcctggggttccttttgtctattccttttttaaaagactagtgtttattttatgtgcatgagtgttttgcatccacattcgcctgtatacacacctggttctgtggaggtcaggagagggtgctggatgccctggcactagagccttggatggttatgtgagcccctgccacaggggagctcagaaccaaatccaggtcctctggaagagcaaccagagctcttaaaacttctaagtatccctccatcccctttccatcatatttggaaaggagaaaactgctacccatgcctggcatttatttcagagattaactgtctgtgtaaaacttgacattgaaagtgcactattctgtttcccattcatacttagttgagactactgtaagtcagttagggctttttttgtttggttccttggttagtttggagtgtgtttgtgagctcattaacaggctttcaatatgtagctggaatttgctgtgtagaccagacaggcctcaaatttgtggcaatcctccctgcatcttcccagaatgccctggtacaggcataaaccaccgtgcccagcagtaaaacaatctggtgaggtattattagtcgtgtgctgtgacccagaaaccccactcctggcaatttactgggaaggaacaaacaaagggctaggggagccatatggcctgcagttagagaaaattagatccaactgaaaaatcaacctaaaggtgtaaaagccaagcagttaagaaactgacaagctcatgatggaagccgaggccatcgtgaacactcttcattttaggccccacgtatcactggggacaactgagagtcaaagtacaggtaaggagaccaaggcttttcaggactcaggctgtctcagtgaaaagcccagaagagcagtaattgaaagagctcagacgatgtgtctgatctcctctgtttgtttgttgctgtattatttccactaacttatttgggaggaaaaaaaacagttcacaggcttcttttcttgaaatactggggattgctgggatcgaacccagggataggtttttagtttctaaaataacatagatcatgccctgtttgctttttggaatatgtttgcgctgcccttattttcatgttcaaatactgctccattttgcgtgactctttagtattggtttgatgatttgcatattagattagattgtatttcagttctcagacttatttatcaattctagttttctctttttgttgttttaaaggactcctgagtatatttcagaactgaaccatttcaaccgagctgaagcattctgccttcctagtggtacctcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcgggaattcggatccagatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgac

SEQ ID NO: 3 - DNA, 25 bp, Artificial Sequence
  misc_feature: Primer
agtaattgaaagagctcagacgatg

SEQ ID NO: 4 - DNA, 23 bp, Artificial Sequence
  misc_feature: Primer
tgtcaccctcttggtcttggtgc

SEQ ID NO: 5 - DNA, 22 bp, Artificial Sequence
  misc_feature: Primer
gtactccacccaagtcgccgtc

SEQ ID NO: 6 - DNA, 23 bp, Artificial Sequence
  misc_feature: Primer
gcagcagcatcgaagcttctcag

SEQ ID NO: 7 - DNA, 48 bp, Artificial Sequence
  misc_feature: Missense
gcagcagcatcgaagcttctcagattttacttccatctggccacctcc

SEQ ID NO: 8 - DNA, 20 bp, Artificial Sequence
  misc_feature: Primer
ccgccaagagccgcctgcag

SEQ ID NO: 9 - DNA, 61 bp, Artificial Sequence
  misc_feature: Primer
gcagcagcatcgaagcttctcagattttacttccacctggccacctcctagtttatgatgg

SEQ ID NO: 10 - DNA, 1152 bp, Homo sapiens
atggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccagatggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtga

SEQ ID NO: 11 - DNA, 1152 bp, Homo sapiens
atggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaactaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtga

SEQ ID NO: 12 - DNA, 19 bp, Artificial Sequence
  misc_feature: Primer
gcattggagacacccccag

SEQ ID NO: 13 - DNA, 21 bp, Artificial Sequence
  misc_feature: Primer
gcttttactgaccatgcgagc

SEQ ID NO: 14 - DNA, 25 bp, Artificial Sequence
  misc_feature: Primer
ctggaagacgaagctgctggtcacg

SEQ ID NO: 15 - DNA, 9990 bp, Artificial Sequence; identical to SEQ ID NO: 2
  misc_feature: PrP/tau transgene construct